(19) United States
(12) Patent Application Publication
(10) Pub. No.: US 2004/0254176 A1
(43) Pub. Date: Dec. 16, 2004

(54) Combination of an ace inhibitor, a calcium channel blocker and a diuretic

(76) Inventors: Melissa Lynn Grigorieff, New York, NY (US); Suraj Shivappa Shetty, Far Hills, NJ (US); Randy Lee Webb, Flemington, NJ (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/466,198

(22) PCT Filed: May 16, 2003
(86) PCT No.: PCT/EP03/05195

Related U.S. Application Data
(60) Provisional application No. 60/381,545, filed on May 17, 2002.

(51) Int. Cl. A61K 31/549; A61K 31/455; A61K 31/401

(52) U.S. Cl. 514/223.5; 514/423; 514/355

(57) ABSTRACT

The present invention relates to a method of treatment of a condition or disease selected from the group consisting of hypertension, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, endothelial dysfunction, cognitive dysfunction (such as Alzheimer's), glaucoma and stroke, comprising administering, to a mammal in need thereof, a therapeutically effective amount of a combination comprising (i) an ACE inhibitor, (ii) a calcium channel blocker (CCB), and (iii) a diuretic.

The present invention relates to a method of treatment of a condition or disease selected from the group consisting of hypertension, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, endothelial dysfunction, cognitive dysfunction (such as Alzheimer's), glaucoma and stroke, comprising administering, to a mammal in need thereof, a therapeutically effective amount of a combination comprising (i) an angiotensin converting enzyme (ACE) inhibitor, (ii) a calcium channel blocker (CCB), and (iii) a diuretic.

The present invention relates to a pharmaceutical composition comprising (i) an (ACE) inhibitor, (ii) a calcium channel blocker (CCB), and (iii) a diuretic, or, where appropriate, in each case a pharmaceutically acceptable salt thereof, especially for the treatment of a disease or condition as set forth hereinbefore or hereinafter.

The invention likewise relates to the use of (i) an angiotensin converting enzyme (ACE) inhibitor, (ii) a calcium channel blocker (CCB), and (iii) a diuretic, or, where appropriate, in each case a pharmaceutically acceptable salt thereof, for the manufacture of a medicament for the treatment of a disease or condition as set forth hereinbefore or hereinafter.

The present invention also relates to a kit of parts comprising

(i) a pharmaceutical composition of an (ACE) inhibitor or a pharmaceutically acceptable salt thereof, (ii) a pharmaceutical composition of a calcium channel blocker (CCB) or a pharmaceutically acceptable salt thereof, and (iii) a pharmaceutical composition of a diuretic, or a pharmaceutically acceptable salt thereof,

in the form of two or three separate units of the components (i) to (iii).

Clinical studies have shown that lowering blood pressure in hypertensive patients reduces mortality and morbidity (Collins R, Peto R, MacMahon S, Hebert P, Fiebach N H, Eberlein K A, Godwin J, Qizilbash N, Taylor J O, Hennekens C H, Lancet 1990, 335(8693):827-38). Despite the availability and use of various classes of agents in the treatment of this medical condition, adequate control of blood pressure is not always achieved (Waeber B, Brunner H R, Am J Hypertens 1997, 10(7 Pt 2):131S-137S). Using a combination of agents is one way to achieve the desired therapeutic end-point. However, arbitrary selection of antihypertensive agents of different classes for inclusion in a combination therapy regimen does not necessarily help achieve target levels of blood pressure in hypertensive mammals including humans (MacGregor G A, Markandu N D, Banks R A, Bayliss J, Roulston J E, Jones J C, Br Med J (Clin Res Ed), 284(6317):693-6).

The National Health and Nutrition Examination Survey (NHANES 3) reported that only half of all hypertensive Americans that were receiving treatment had their blood pressure controlled to <140/90 mmHg. Many reasons exist for inadequate blood pressure control, including poor patient compliance, reluctance of physicians to titrate medication, concerns with adverse events and lack of success with monotherapy or even dual therapy. Recent studies have shown that most patients require a combination of antihypertensive medications to reach goal blood pressure. In addition, there has been increased emphasis on the need for aggressive treatment of hypertension to avoid cardiovascular complications. For certain patient groups, including diabetics and patients with renal disease, a target blood pressure of <130/80 mmHg has been recommended. An average of 3 antihypertensive medications were required to achieve this level of blood pressure control in several large studies.

The combination of an ACE-inhibitor, calcium channel blocker, and diuretic provides unique advantages in terms of both efficacy and safety because the mechanism of action of the 3 drugs are all complementary. This results in significant blood pressure lowering to aggressive target goals, along with a reduction in side effects seen with mono- or dual therapies. A diuretic results in volume depletion and smooth muscle relaxation. The volume depletion activates the renin-angiotensin system, which is blocked by an ACE-inhibitor. One result would be less hypokalemia seen with the diuretic, and the diuretic, correspondingly, would reduce hyperkalemia that is sometimes observed with an ACE-inhibitor. CCB is a direct-acting arterial vasodilator. which can lead to a compensatory activation of the sympathetic nervous system. Blockade of the renin angiotensin system, through ACE inhibition, attenuates the overactivity of the sympathetic nervous system via attenuation of neurotransmitter release from sympathetic neurons. ACE-inhibitors act as arterial and venous vasodilators. The result is less CCB induced edema due to ACE-inhibitor-induced post-capillary dilation acting to offset the pre-capillary dilation elicited by the CCB. Furthermore, the edematous state is partially mitigated by the actions of the diuretic. Thus, the triple combination provides unique complementary benefits for both efficacy and safety/tolerability. The triple combination allows for aggressive control of hypertension with minimal side-effects in single once-daily administration.

### EXAMPLE OF A DESIGN OF A CLINICAL TRIAL

Dose-ranging multifactorial study using a minimum of two doses of each agent in patients with moderate to severe hypertension (systolic BP 160-200, diastolic BP 100-119), all ages and all racial groups. The study design utilizes a double blind, placebo-controlled format. A one-three week placebo run-in followed by 8 weeks of double-blind treatment.

Primary efficacy variable: reduction in diastolic BP

Secondary efficacy variable: reduction in systolic BP, responder rate (% of patients achieving target BP), comparison of adverse events with triple combination vs. mono and dual therapies.

The present invention also relates to a combination of pharmaceutically active organic compounds with different modes of action for effecting blood pressure-lowering, and for attenuating the varied pathological sequelae of hypertension and several other cardiovascular disorders, e.g. left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, endothelial dysfunction, cognitive dysfunction (such as Alzheimer's), glaucoma and stroke.

Furthermore, this invention addresses the unequal responsiveness of humans to antihypertensive monotherapy, based on age and/or ethnicity (Campo C, Segura J, Ruilope L M, J Clin Hypertens (Greenwich) January 2002, 4(1):35-40).

ACE inhibitors to be employed in the in the present invention are selected from the group consisting of alacepril, benazepril, benazeprilat, captopril, ceronapril, cilazapril, delapril, enalapril, enalaprilat, fosinopril, imidapril, lisinopril, moexipril, moveltopril, perindopril, quinapril, quinaprilat, ramipril, ramiprilat, spirapril, temocapril, trandolapril, and zofenopril or, in each case, a pharmaceutically acceptable salt thereof.

Preferred ACE inhibitors are those agents that have been marketed, most preferred are benazepril, benazaprilat, ramipril and ramiprilat, quinapril and quinaprilat, lisinopril, trandolapril, enalapril and enalaprilat.

CCBs useful in the present invention are e.g. amlodipine, felodipine, isradipine, lacidipine, lercanidipine, nicardipine, nifedipine, niguldipine, niludipine, nimodipine, nisoldipine, nitrendipine, nivaldipine, and ryosidine, which all belong to the group of dihydropyridines (DHPs). Also useful are non-DHP CCBs like anipamil, diltiazem, fendiline, flunarizine, gallopamil, mibefradil, prenylamine, tiapamil and verapamil. These DHP and non-DHP CCBs also include their pharmaceutically acceptable salts.

Preferred CCBs are amlodipine, for example the besylate or the maleate salt, and felodipine.

The diuretic that, according to the present invention, is used in combination with the ACE inhibitor and the CCB is preferably selected from the group consisting of bumetanide, ethacrynic acid, furosemide, torsemide, amiloride, spironolactone, triamterene, chlorothalidone, chlorothiazide, hydrochlorothiazide (HCTZ), hydroflumethiazide, methylchlorothiazide, metolazone, and dichlorphenamide. Diuretics may be understood in three classes: thiazides (e.g., HCTZ), potassium sparing (e.g., triamterene, spironolactone and eplerenone) and “loop” diuretics (e.g., furosemide). A further diuretic is amiloride.

The most preferred diuretic for the intended combination is a thiazide diuretic, e.g. hydrochlorothiazide.

The structure of the active agents identified by generic or tradenames may be taken from the actual edition of the standard compendium “The Merck Index” or from databases, e.g. LifeCycle Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The invention provided for unexpected and surprising results regarding the effects of the combination of (i) an ACE inhibitor, (ii) an CCB and (iii) a diuretic in the treatment of hypertension, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, endothelial dysfunction, cognitive dysfunction (such as Alzheimer's), glaucoma and stroke in mammals.

In the method of treatment according to the present invention the peripheral arteriolar vasodilation following treatment with a CCB was found to be complementary to an ACEI inhibitor which dilates both the arterial and venous sides of the vascular tree. This arterial and venous action has been shown to resolve the edema that may result from administration of the CCB alone. Also, an activation of the renin-angiotensin-aldosterone system (RAAS) and the consequent pressure and volume-retaining effects is, at least partly, negated by inhibiting the synthesis of angiotensin II due to treatment with the ACE inhibitor. Also, the volume depleting effects of the diuretic provides an additional blood pressure lowering effect. Recent results of the ALLHAT trial demonstrated that the use of a diuretic is a preferred antihypertensive treatment. Consequently, the addition of a diuretic to a CCB and an ACE inhibitor might produce further unexpected benefit.

The inclusion of a CCB into the dual combination of an ACE inhibitor and a diuretic very surprisingly increased the net responder rate and, in addition, a reflex activation of the sympathetic nervous system, a frequent side effect of the treatment with CCBs, is unexpectedly suppressed to a large extent.

Thus it was unexpectedly and also very surprisingly found that the inventive method of treatment of a condition or disease selected from the group consisting of hypertension, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, endothelial dysfunction, cognitive dysfunction (such as Alzheimer's), glaucoma and stroke, comprising administering, to a mammal in need thereof, a therapeutically effective amount of a combination comprising

(i) an ACE inhibitor selected from the group consisting of alacepril, benazepril, benazeprilat, captopril, ceronapril, cilazapril, delapril, enalapril, enalaprilat, fosinopril, imidapril, lisinopril, moexipril, moveltopril, perindopril, quinapril, quinaprilat, ramipril, ramiprilat, spirapril, temocapril, trandolapril, and zofenopril or, in each case, a pharmaceutically acceptable salt thereof, (ii) a calcium channel blocker (CCB) slected from the group consisting of amlodipine, felodipine, isradipine, lacidipine, nicardipine, nifedipine, niguldipine, niludipine, nimodipine, nisoldipine, nitrendipine, nivaldipine, ryosidine, anipamil, diltiazem, fendiline, flunarizine, gallopamil, mibefradil, prenylamine, tiapamil and verapamil or, in each case, a pharmaceutically acceptable salt thereof, and (iii) a diuretic selected from the group consisting of bumetanide, ethacrynic acid, furosemide, torsemide, amiloride, spironolactone, eplerenone, triamterene, chlorothalidone, chlorothiazide, hydrochlorothiazide, hydroflumethiazide, methylchlorothiazide, metolazone, and dichlorphenamide and also amiloride achieves greater therapeutic effect than a monotherapy with only one of the above compounds. The greater therapeutic efficacy is also achieved with respect to the treatment of the conditions named herein with a dual combination like e.g. a combination of an ACE inhibitor with a CCB or a combination of an ACE inhibitor with a diuretic.

Greater efficacy achieved according to the present invention can further be documented as a prolonged duration of action. The duration of action can be monitored as either the time to return to baseline prior to the next dose or as the area under the curve (AUC) and is expressed as the product of the change in blood pressure in millimeters of mercury (change in mmHg) and the duration of the effect (minutes, hours or days). The aforementioned combination treatment also unexpectedly reduces blood pressure in hypertensive mammals in a smooth and sustained fashion. The trough:peak blood pressure ratio demonstrated by this combination is close to unity leading to a more homogenous blood pressure control during the inter-dosing period. The combined regimen of an ACE inhibitor, a CCB and a diuretic, or in each case a pharmaceutically acceptable salt thereof, in particular the combination of benazepril, especially the hydrochloride thereof, amlodipine, especially the maleate or more preferred the besylate thereof, and hydrochlorothiazide (HCTZ) is, at least in part, devoid of either orthostatic hypotension or first-dose hypotension, and no incidences of rebound hypertension occur after cessation of treatment. It can be shown that in particular combination therapy with benazepril, amlodipine, and HCTZ, more preferably benazepril hydrochloride, amlodipine besylate and HCTZ, results in lessening of pulse pressure in hypertensive mammals. Therefore, the combination of benazepril, amlodipine and HCTZ, more preferably benazepril hydrochloride, amlodipine besylate and HCTZ, is a particularly preferred combination in the context of the present invention.

Furthermore, combination therapy with an ACE inhibitor, a CCB and a diuretic can ameliorate endothelial dysfunction and improve vascular compliance and distensibility in hypertensive mammals. It can also slow the progression of cardiac, renal and cerebral end-organ damage in these mammals. Further benefits are that lower doses of the individual drugs to be combined according to the present invention can be used to reduce the dosage, for example, that the dosages need not only often be smaller but are also applied less frequently, or can be used to diminish the incidence of side effects. Surprisingly, the combination significantly reduced the incidences of peripheral edema relative to those observed in mammals treated with a CCB e.g. amlodipine alone. Also, the undesirable effects of diuretics e.g. HCTZ on serum lipids, glucose, and uric acid levels were surprisingly attenuated in mammals treated with the combined regimens of benazepril, amlodipine and HCTZ.

In particular the combined administration of benazepril or a pharmaceutically acceptable salt thereof, amlodipine or a pharmaceutically acceptable salt thereof, and HCTZ results in a significant response in a greater percentage of treated patients, that is, a greater responder rate results, regardless of the underlying etiology of the condition. This is in accordance with the desires and requirements of the patients to be treated. The combination treatment effectively lowered blood pressure in hypertensive patients in all age groups including pre and postmenopausal women. It can be shown that combination therapy with benazepril, amlodipine, and HCTZ results in a more effective antihypertensive therapy (whether for malignant, essential, reno-vascular, diabetic, isolated systolic, or other secondary type of hypertension) and lessening of pulse pressure through improved efficacy. The combination is also useful in the treatment or prevention of left ventricular dysfunction and hypertrophic cardiomyopathy, diabetic cardiac myopathy, supraventricular and ventricular arrhythmias, atrial fibrillation, atrial flutter or detrimental vascular remodeling. It can further be shown that a benazepril, amlodipine, and HCTZ combination therapy proves to be beneficial in the treatment and prevention of myocardial infarction and its sequelae. A benazepril, amlodipine, and HCTZ combination is also useful in treating atherosclerosis, angina (whether stable or unstable), renal insufficiency (diabetic and non-diabetic), peripheral vascular disease, cognitive dysfunction, and stroke. Furthermore, the improvement in endothelial function with the combination therapy using benazepril, amlodipine, and HCTZ, more preferably benazepril hydrochloride, amlodipine besylate and HCTZ, provides benefit in diseases in which normal endothelial function is disrupted such as heart failure, angina pectoris and diabetes. Furthermore, the combination of the present invention may be used for the treatment or prevention of secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, endothelial dysfunction, cognitive dysfunction (such as Alzheimer's), glaucoma and stroke. The combination regimen also surprisingly reduces the rate of progression of cardiac, renal and cerebral end-organ damage. Use of lower doses of the individual drugs in the combination regimen can be used to diminish the incidence of side effects such as cough and angioedema that are often associated with the use of ACE inhibitors. By providing enhanced efficacy, safety and tolerability, the combination of drugs indicated in this invention also has the potential to promote patient compliance, a major consideration in the pharmacological treatment of hypertension.

Very surprisingly the effects of the combination according to the present invention may also allow for elevated dosages of the ACE inhibitor, the CCB and the diuretic, respectively, without causing intolerable side effects. This particularly applies to the dosage of the CCB amlodipine. Currently, the highest daily dose allowed is 10 mg amlodipine. In the present combination daily dosages of amlodipine up to 60 mg may be administered without more side effects than a daily dosage of 5 or 10 mg amlodipine.

The daily dosage of the ACE inhibitor is, according to the invention, between 0.5 and 80 mg daily, preferably between 5 and 60 mg, e.g. 20, 40 or 60 mg.

The daily dosage of the CCB in the inventive combination is between 1 and 60 mg and preferably between 2.5 and 40 mg, e.g. 2.5, 5, 10, 20, 30 or 40 mg.

Finally, the daily dosage of the diuretic in the inventive combination is between 5 and 200 mg, preferably between 5 and 100 mg and more preferably between 5 and 50 mg.

All of the daily dosages given above are only generally referred to in the context of the present invention and may however vary in range depending on the actual ACE inhibitor, CCB and diuretic actually employed in the combination.

In particular, the preferred combination of benazepril, amlodipine and hydrochlorothiazide advantageously contain between 5 and 80 mg benazepril, e.g. 5, 10, 20, 40, 60, or 80 mg benazepril, wherein the indicated amounts of benazepril or benazeprilat are understood to be amounts given in benazepril hydrochloride equivalents, irrespective of the actual salt form used. Examples of preferred ranges of benazepril are 2.5-7.5 mg, 7.5-12.5 mg, 12.5-17.5 mg, 17.5-22.5 mg 22.5-27.5 mg 27.5-32.5 mg, 32.5-40 mg, 40-50 mg, 50-65 or 65-80.

The preferred amount of amlodipine in said combination is between 2.5 and 60 mg, e.g. 2.5, 5, 7.5, 10, 15, 20, 30 or 40 mg, more preferred between 2.5 and 20 mg or 2.5 and 10 mg.

The amlodipine dosages set forth herein are understood to be amlodipine free base equivalents, irrespective of the salt form used. Examples of preferred ranges of amlodipine are 2-8 mg, 8-12 mg, 12-18 mg or 18-22 mg.

Finally, the amount of hydrochlorothiazide or HCTZ contained in this preferred combination ranges preferably from 5 to 100 mg, more preferred from 5 to 50 mg or 5 to 25 mg, e.g. 6.25, 12.5, 25 or 40 mg. Examples of preferred ranges of HCTZ are 5-10 mg, 10-19 mg, 19-29 mg, 29-39 mg or 39-50 mg.

In the following table examples for particularly preferred combinations of benazepril, amlodipine and HCTZ are given:

| benazepril | amlodipine | HCTZ |
|---|---|---|
| 5 mg | 10 mg | 6.25 mg |
| 10 mg | 2.5 mg | 6.25 mg |
| 10 mg | 2.5 mg | 12.5 mg |
| 10 mg | 2.5 mg | 25 mg |
| 10 mg | 5 mg | 12.5 mg |
| 10 mg | 5 mg | 25 mg |
| 10 mg | 10 mg | 12.5 mg |
| 10 mg | 10 mg | 25 mg |
| 15 mg | 2.5 mg | 6.25 mg |
| 15 mg | 5 mg | 6.25 mg |
| 20 mg | 2.5 mg | 12.5 mg |
| 20 mg | 5 mg | 12.5 mg |
| 20 mg | 5 mg | 25 mg |
| 20 mg | 10 mg | 12.5 mg |
| 20 mg | 10 mg | 25 mg |
| 30 mg | 2.5 mg | 12.5 mg |
| 30 mg | 2.5 mg | 25 mg |
| 30 mg | 5 mg | 12.5 mg |
| 30 mg | 5 mg | 25 mg |
| 40 mg | 5 mg | 12.5 mg |
| 40 mg | 5 mg | 25 mg |
| 40 mg | 10 mg | 12.5 mg |
| 40 mg | 20 mg | 25 mg |
| 40 mg | 20 mg | 40 mg |
| 60 mg | 10 mg | 12.5 mg |
| 60 mg | 10 mg | 25 mg |
| 60 mg | 10 mg | 40 mg |
| 80 mg | 5 mg | 12.5 mg |
| 80 mg | 5 mg | 25 mg |
| 80 mg | 5 mg | 40 mg |
| 80 mg | 10 mg | 12.5 mg |
| 80 mg | 10 mg | 25 mg |
| 80 mg | 10 mg | 40 mg |

The combination of an ACE inhibitor, a CCB and a diuretic to be used in the method of the present invention will generally be present in the form of a combined pharmaceutical composition. The active ingredients of the combination as disclosed herein may alternatively be used for simultaneous or sequential administration in any order, for separate administration or, most preferably, as a fixed combination.

Another example of a preferred combination, comprises an amount of benazepril between 2.5 and 12.5 mg (e.g. 5 mg or 10 mg), an amount of amlodipine between 2 and 8 mg (e.g. 2.5 mg or 5 mg) and an amount of HCTZ between 5 and 30 (e.g. 6.25 mg, 12.5 mg or 25 mg), preferably between 5 and 16 (e.g. 6.25 mg or 12.5 mg).

A further example of a preferred combination, comprises an amount of benazepril between 17.5 and 22.5 mg (e.g. 20 mg), an amount of amlodipine between 2 and 8 mg (e.g. 2.5 mg or 5 mg) and an amount of HCTZ between 10 and 30 (e.g. 12.5 mg or 25 mg).

Another example of a preferred combination, comprises an amount of benazepril between 12.5 and 30 mg, an amount of amlodipine between 2 and 8 mg and an amount of HCTZ between 5 and 30 (e.g. a tablet of Lotrel® 2.5 mg of amlodipine and 10 mg of benazepril, and a tablet of Cibadrex® 10 mg of benazepril and 12.5 mg of HCTZ).

Fixed dose combinations of amlodipine besylate and benazepril hydrochloride are being marketed under the trade name Lotrel®. Corresponding amounts of the active ingredients are 2.5 mg of amlodipine and 10 mg of benazepril, 5 mg of amlodipine and 10 mg of benazepril, and 5 mg of amlodipine and 20 mg of benazepril, the amounts of amlodipine corresponding to the free base and the amounts of benazepril corresponding to the hydrochloride. As used herein, the term “Lotrel® combination” refers to these dosage combinations.

Fixed dose combinations of benazepril and hydrochlorothiazide are being marketed under the trade name Cibadrex® and Lotensin HCT®. Corresponding amounts of the active ingredients are 5 mg of benazepril and 6.25 mg of HCTZ, 10 mg of benazepril and 12.5 mg of HCTZ, 20 mg of benazepril and 12.5 mg of HCTZ, and 20 mg of benazepril and 25 mg of HCTZ, respectively. The amount of benazepril in these combinations is the amount of the hydrochloride. The term “Cibadrex® combination”, as used herein, designates these doasage combinations.

Benazepril is commercially available under the trade name Cibacen® or Lotensin® and marketed in three different dosage forms containing 5, 10 and 20 mg benazepril hydrochloride, respectively.

Amlodipine is commercially available under the trade name Norvasc®. It is marketed in two different dosage forms containing amlodipine besylate in amount to 5 and 10 mg of the free base of amlodipine, respectively.

While the ingredients of the combination according to the present invention can be administered at different times, they are most preferably administered at the same time. Most conveniently, this is via a single, fixed combination dosage form. However, the CCB can be administered at times different from the administration of the ACE inhibitor and the diuretic and the invention benefits still be realized. When administered at different times, the CCB, the diuretic and the ACE inhibitor should be given within about 16 hours of each other, preferably within about 12 hours of each other, more preferably within about 8 hours of each other, most preferably within about 4 hours of each other. Of course, these time periods can be extended if the dosage form is one that will “administer” the agents for extended periods.

When the CCB, the diuretic and the ACE inhibitor are given substantially simultaneously, they may be given by a single fixed combination dosage form or by different dosage forms, whichever are convenient. When given by different dosage forms, it is irrelevant whether the route of administration is the same for each agent or different for each agent Any route of administration known for the individual agents is acceptable for the practice of the present invention. Most preferably, the agents are given in a fixed combination, or at least substantially simultaneously, i.e. within about 1 hour of each other. Also, the most suitable dosage form is an oral dosage form, where an oral administration is a clinically suitable route.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is more beneficial than the effect that would be obtained by use of only any one of the components.

Thus the present invention also relates to a kit of parts comprising

(i) a pharmaceutical composition of an (ACE) inhibitor or a pharmaceutically acceptable salt thereof,

(ii) a pharmaceutical composition of a calcium channel blocker (CCB) or a pharmaceutically acceptable salt thereof, and

(iii) a pharmaceutical composition of a diuretic, or a pharmaceutically acceptable salt thereof, in the form of two or three separate units of the components (i) to (iii).

Dosages of the agents of the combination of the present invention include all dosages at which the agents are used individually. Corresponding dosages for other salts of amlodipine, for free benazepril and other salts of benazepril, and benazeprilat and its salts will be readily apparent to those of ordinary skill in the art. In each of the dosages set forth here, the range is the acceptable range based on adult mammal of approximately 50 to about 70 kg. Modified dosage ranges for mammals of other sizes and stages of development will be apparent to those of ordinary skill in the art.

Benazepril and amlodipine are normally physically incompatible substances. Hence, if incorporated into a single dosage form they must be kept physically separated. This may be accomplished in any of the myriad ways known in the art, such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc. Due to the incompatibility, combination products of the two agents in an injectable solution may not really be acceptable. For convenience purposes, a coated compressed tablet of benazepril together with amlodipine powder in a capsule has been found to be the most desirable oral form.

For the present purposes, preferred mammals are rabbits, dogs, goats, hogs, sheep, horses, cattle, and primates, more preferably primates, most preferably humans.

The person skilled in the pertinent art is fully enabled to select a relevant test model to prove the efficacy of a combination of the present invention in the herein before and hereinafter indicated therapeutic indications.

The advantage of the present combination is, for example, demonstrated in a clinical study or in the test procedure as essentially described hereinafter. Many clinical study protocols adapted for our combinations are known by the person skilled in the art. An example of a clinical trial protocol useful to demonstrate the unexpected advantages of our new combinations is described by to Messerli F H et al. (Am J Hypertens June 2002 15(6):550-6). The same protocol is performed with our preferred combinations such as described herein. This protocol is hereby incorporated into the present application by reference to this publications.

Representative studies are carried out with a combination of benazepril, amlodipine, and HCTZ applying the following methodology. Drug efficacy is assessed in various animal models including the deoxycorticosterone acetate-salt rat (DOCA-salt), the Dahl salt-sensitive rat (DSS and the control salt-resistant; DSR) and the spontaneously hypertensive rat (SHR), either maintained on a normal salt diet or with salt loading (4-8% salt in rat chow or 1% NaCl as drinking water).

The DOCA-salt test model utilizes either an acute or chronic study protocol. An acute study procedure involves assessment of the effects of various test substances over a six-hour experimental period using rats with indwelling femoral arterial and venous catheters. The Acute Study Procedure evaluates test substances for their ability to reduce blood pressure during the established phase of DOCA-salt hypertension. In contrast, the Chronic Study Procedure assesses the ability of test substances to prevent or delay the rise in blood pressure during the development phase of DOCA-salt hypertension. Therefore, blood pressure will be monitored in the chronic study procedure by means of a radiotransmitter (M. K. Bazil, C. Krulan and R. L. Webb. Telemetric monitoring of cardiovascular parameters in conscious spontaneously hypertensive rats. J. Cardiovasc. Pharmacol. 22: 897-905, 1993). The radiotransmitter is surgically implanted into the abdominal aorta of rats, prior to the initiation of DOCA-salt treatment and thus, prior to the induction of hypertension. Blood pressure is chronically monitored for periods of up 6 weeks (approximately one week prior to DOCA-salt administration and for 5 weeks thereafter).

Rats are anesthetized with 2-3% isoflurane in oxygen inhalant followed by Amytal sodium (amobarbital) 100 mg/kg, ip. The level of anesthesia is assessed by a steady rhythmic breathing pattern.

Acute Study Procedure:

Rats undergo a unilateral nephrectomy at the time of DOCA implantation. Hair is clipped on the left flank and the back of the neck and scrubbed with sterile alcohol swabs and povidone/iodine. During surgery rats are placed on a heating pad to maintain body temperature at 37° C.

A 20 mm incision is made through the skin and underlying muscle to expose the left kidney. The kidney is freed of surrounding tissue, exteriorized and two ligatures (3-0 silk) are tied securely around the renal artery and vein proximal to their juncture with the aorta. The renal artery and vein are then severed and the kidney removed. The muscle and skin wounds are closed with 4-0 silk suture and stainless steel wound clips, respectively. At the same time, a 15 mm incision is made on the back of the neck and a 3-week-release pellet (Innovative Research of America, Sarasota, Fla.) containing deoxycorticosterone acetate (100 mg/kg) is implanted subcutaneously. The wound is then closed with stainless-steel clips and both wounds are treated with povidone/iodine; the rats are given a post-surgical intramuscular injection of procaine penicillin G (100,000 U) and buprenorphine (0.05-0.1 mg/kg) s.c. The rats are immediately placed on 1% NaCl+0.2% KCl drinking water; this treatment continues for at least 3 weeks at which time the animals have become hypertensive and available for experimentation.

Forty-eight hours prior to experimentation, animals are anesthetized with isoflurane and catheters are implanted in the femoral artery and vein for measuring arterial pressure, collection of blood, and administration of test compounds. Rats are allowed to recover for 48 hours while tethered in a Plexiglas home cage, which also serves as the experimental chamber.

Chronic Study Procedure:

This procedure is the same as above except that rats are implanted with a radiotransmitter, 7-10 days prior to the unilateral nephrectomy and initiation of DOCA and salt. In addition, rats do not undergo surgery for placement of femoral arterial and venous catheters. Radiotransmitters are implanted as described in M. K. Bazil, C. Krulan and R. L. Webb. Telemetric monitoring of cardiovascular parameters in conscious spontaneously hypertensive rats. J.Cardiovasc. Pharmacol. 22: 897-905, 1993.

Protocols are then set-up on the computer for measurement of blood pressure, heart rate, etc, at predetermined time points. Baseline data is collected at various time points and over various time intervals. For example, baseline or pre-dose values usually consist of data collection and averaging over 3 consecutive, 24-hour time periods prior to drug administration.

Blood pressure, heart rate and activity are determined at various pre-selected time points before, during, and after drug administration. All measurements are performed in unrestrained and undisturbed animals. The maximum study time, determined by battery life, could be as long as nine months. For studies of this duration, rats are dosed orally (1-3 ml/kg vehicle), no more than twice daily or drug is administered via the drinking water or mixed with food. For studies of a shorter duration, that is, up to 8 weeks, drugs are given via subcutaneously implanted osmotic minipumps. Osmotic minipumps are selected based on drug delivery rate and time. Benazepril dosages range from 1 to 100 mg/kg/day, amlodipine dosages range from 1 to 75 mg/kg/day, and HCTZ dosages range from 1 to 75 mg/kg/day. Additionally, SHR are utilized to study the effects of benazepril in combination with amlodipine, and HCTZ. The hypertensive background of the SHR is modified either by chronic salt loading in an effort to suppress the RAAS or chronic salt depletion to activate the RAAS in the SHR. These manipulations will be carried out to more extensively evaluate the efficacy of the various test substances. Experiments are performed in spontaneously hypertensive rats (SHR) supplied by Taconic Farms, Germantown, N.Y. (Tac:N(SHR)fBR). A radiotelemetric device (Data Sciences International, Inc., St. Paul, Minn.) is implanted into the lower abdominal aorta of all test animals between the ages of 14 to 16 weeks of age. All SHR are allowed to recover from the surgical implantation procedure for at least 2 weeks prior to the initiation of the experiments. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed SHR in their home cages. The arterial blood pressure and heart rate are measured every 10 minutes for 10 seconds and recorded. Data reported for each rat represent the mean values averaged over a 24 hour period and are made up of the 144-10 minute samples collected each day. The baseline values for blood pressure and heart rate consist of the average of three consecutive 24 hour readings taken prior to initiating the drug treatments. All rats are individually housed in a temperature and humidity controlled room and are maintained on a 12 hour light dark cycle. A typical experimental design for the determination of the effects of the triple combination are in essence identical to the clinical study design. A factorial design is utilized in which at least two doses of each of the agents is compared to that of either the monotherapy of the dual combination therapy over the course of three to six weeks of drug treatment. The use of a factorial design allows for a detailed statistical analysis to be used, including a response-surface analysis. For example, a fixed dose combination with valsartan and amlodipine in the SHR was performed (R. L. Webb, N. Yao, M. Thoma and M. de Gasparo. Chronic effects of valsartan with amlodipine on blood pressure and cardiac mass in spontaneously hypertensive rats (SHR). J Hypertension 18(Suppl. 4):S80, 2000).

In addition to the cardiovascular parameters, weekly determinations of body weight also are recorded in all rats. Treatments are administered in the drinking water, via daily oral gavage or in osmotic minipumps as stated above. If given in drinking water, water consumption is measured five times per week. Benazepril, especially the hydrochloride thereof, amlodipine, especially the besylate thereof, and HCTZ doses for individual rats are then calculated based on water consumption for each rat, the concentration of drug substance in the drinking water, and individual body weights. All drug solutions in the drinking water are made up fresh every three to four days. Typical dosages for benazepril in drinking water range from 1 to 100 mg/kg/day, dosages of amlodipine range from 1 to 75 mg/kg/day, and dosages of HCTZ range from 1 to 75 mg/kg/day. In most situations, a daily dose will not exceed 100 mg/kg/day when administered as the monotherapy. In combination, lower dosages of each agent are used and correspondingly, benazepril is given in the range of 1 to 30 mg/kg/day, and amlodipine and HCTZ are give in dosages below 50 mg/kg/day. However, in cases wherein the responder rate is increased with combination treatment, the dosages are identical to those used as monotherapy.

When drugs are administered by oral gavage, the dose of benazepril ranges from 1 to 50 mg/kg/day and that of amlodipine and HCTZ does not exceed 75 mg/kg/day. Upon completion of the chronic studies, SHR or DOCA-salt rats are anesthetized, blood samples obtained for biochemical analysis and the heart rapidly removed. After separation and removal of the atrial appendages, left ventricle and left plus right ventricle (total) are weighed and recorded. Left ventricular and total ventricular mass are then normalized to body weight and reported.

Vascular function and structure are evaluated after treatment to assess the beneficial effects of the combination. SHR are studied according to the methods described by Intengan H D, Thibault G, Li J S, Schiffrin E L, Circulation 1999, 100 (22): 2267-2275. Similarly, the methodology for assessing vascular function in DOCA-salt rats is described in Intengan H D, Park J B, Schiffrin, E L, Hypertension, 1999, 34(4 Part 2): 907-913. Assessment of vascular compliance and distensibility following treatment with the combination regimen is performed according to the methods described by Ceiler D L, Nelissen-Vrancken H J, De Mey J G, Smits J F, J Cardiovasc Pharmacol 1998, 31(4):630-7. Amelioration of cardiac, renal, and cerebral injury secondary to hypertension is assessed after treatment with the combination regimen in salt-loaded stroke-prone spontaneously hypertensive rats according to the methods described by Nagura J, Yamamoto M, Hui C, Yasuda S, Hachisu M, Konno F, Clin Exp Pharmacol Physiol 1996, 23(3):229-35. Propensity of the combination therapy to elicit postural or orthostatic hypotension is assessed in SHRs by the methods described by Nabata H, Aono J, Ishizuka N, Sakai K, Arch Int Pharmacodyn Ther 1985, 277(1):104-18. Tendency to produce peripheral edema by the combination regimen was assessed by the methods described by Lacolley P, Poitevin P, Koen R, Levy B I, J Hypertens 1998, 16(3):349-55.

A therapeutically effective amount of each of the component of the combination of the present invention may be administered simultaneously or sequentially and in any order. The corresponding active ingredient or a pharmaceutically acceptable salt thereof may also be used in form of a hydrate or include other solvents used for crystallization. The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions.

The invention also relates to combining separate pharmaceutical compositions in kit form. That is a kit combining three separate units: a benazepril pharmaceutical composition, an amlodipine pharmaceutical composition, and a HCTZ pharmaceutical composition. The kit form is particularly advantageous when the separate components must be administered in different dosage forms (e.g. parenteral benazepril formulation and oral amlodipine or HCTZ formulations) or are administered at different dosage intervals.

In a preferred embodiment, the (commercial) product is a commercial package comprising as active ingredients the combination according to the present invention (in the form of two or three separate units of the components (i) to (iii)), together with instructions for its simultaneous, separate or sequential use, or any combination thereof, in the delay of progression or treatment of the diseases mentioned herein. A preferred commercial package, is where the ACE inhibitor (i) and the diuretic (iii) are present in the form of CIBADREX® or where the ACE inhibitor (i) and the CCB (ii) are present in the form of LOTREL®, or where the ACE inhibitor (i), the CCB (ii) and the diuretic (iii) are present in the form of LOTREL® and CIBADREX®.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition. Preferred dosages for the active ingredients of the pharmaceutical combination according to the present invention are therapeutically effective dosages, especially those that are commercially available.

Benazepril is supplied in the form of suitable dosage unit form, for example, a capsule or tablet, and comprising a therapeutically effective amount, e.g. from about 5 to about 60 mg of benazepril which may be applied to patients. The application of the active ingredient may occur up to three times a day, starting e.g. with a daily dose of 5 mg of benazepril, increasing via 5 mg daily and further to 20 mg daily up to 40 or 60 mg daily. Preferably, benazepril is applied once a day or twice a day in heart failure patients with a dose of 40 mg or 20 mg, respectively, each. Corresponding doses may be taken, for example, in the morning, at mid-day or in the evening. Preferred is q.d. or b.i.d. administration in heart failure.

In case of amlodipine, preferred dosage unit forms are, for example, tablets or capsules comprising e.g. from about 1 mg to about 20 mg, preferably 2.5 to 10 mg daily when administered orally.

In case of HCTZ, preferred dosage unit forms are, for example, tablets or capsules comprising e.g. the amount as set forth herein before, administered orally once a day.

The above doses encompass a therapeutically effective amount of the active ingredients of the present invention.

As used throughout the specification and in the claims, the term “treatment” embraces all the different forms or modes of treatment as known to those of the art and in particular includes preventive, curative and palliative treatment.

1-26. (deleted).

27. A pharmaceutical composition for oral administration comprising:
(i) amlodipine in free or pharmaceutically acceptable salt form in an amount corresponding to from 2.5 mg to 20 mg of amlodipine free base;
(ii) benazepril in free or pharmaceutically acceptable salt form in an amount corresponding to from 5 mg to 80 mg of benazepril hydrochloride; and
(iii) hydrochlorothiazide in an amount of from 5 mg to 25 mg
wherein the amlodipine and benazepril are physically separate from each other.

28. The pharmaceutical composition of claim 27 wherein amlodipine is amlodipine besylate.

29. The pharmaceutical composition of claim 28 wherein benazepril is benazepril hydrochloride

30. The pharmaceutical composition of claim 29 comprising: from 5 mg to 10 mg of amlodipine besylate, from 20 mg to 40 mg of benazepril hydrochloride and from 12.5 mg to 25 mg of hydrochlorothiazide.

31. A pharmaceutical composition for oral administration comprising:
(i) 5 mg of amlodipine besylate;
(ii) 20 mg of benazepril hydrochloride; and
(iii) 12.5 mg of hydrochlorothiazide
wherein the amlodipine and benazepril are physically separate from each other.

32. A pharmaceutical composition for oral administration comprising:
(i) 5 mg of amlodipine besylate;
(ii) 40 mg of benazepril hydrochloride; and
(iii) 12.5 mg of hydrochlorothiazide
wherein the amlodipine and benazepril are physically separate from each other.

33. A pharmaceutical composition for oral administration comprising:
(i) 10 mg of amlodipine besylate;
(ii) 40 mg of benazepril hydrochloride; and
(iii) 12.5 mg of hydrochlorothiazide
wherein the amlodipine and benazepril are physically separate from each other.

34. A pharmaceutical composition for oral administration comprising:
(i) 10 mg of amlodipine besylate;
(ii) 40 mg of benazepril hydrochloride; and
(iii) 25 mg of hydrochlorothiazide
wherein the amlodipine and benazepril are physically separate from each other.

35. A method of treating a hypertensive patient who does not reach a desired target blood pressure following (i) administration of a calcium channel blocker, an ACE inhibitor or diuretic alone, or (ii) administration of a combination of an ACE inhibitor and diuretic or a combination of an ACE inhibitor and calcium channel blocker, comprising administering once a day to said patient a pharmaceutical composition comprising:
(i) amlodipine in free or pharmaceutically acceptable salt form in an amount corresponding to from 2.5 mg to 20 mg of amlodipine free base;
(ii) benazepril in free or pharmaceutically acceptable salt form in an amount corresponding to from 5 mg to 80 mg of benazepril hydrochloride; and
(iii) hydrochlorothiazide in an amount of from 5 mg to 25 mg
wherein the amlodipine and benazepril are physically separate from each other.

36. The method of claim 35 wherein amlodipine is amlodipine besylate.

37. The method of claim 36 wherein benazepril is benazepril hydrochloride.

38. The method of claim 37 wherein the pharmaceutical composition comprises from 5 mg to 10 mg of amlodipine besylate, from 20 mg to 40 mg of benazepril hydrochloride and from 12.5 mg to 25 mg of hydrochlorothiazide.

39. A method of treating a hypertensive patient who does not reach a desired target blood pressure following (i) administration of a calcium channel blocker, an ACE inhibitor or diuretic alone, or (ii) administration of a combination of an ACE inhibitor and diuretic or a combination of an ACE inhibitor and calcium channel blocker, comprising administering once a day to said patient a pharmaceutical composition comprising:
(i) 5 mg of amlodipine besylate;
(ii) 20 mg of benazepril hydrochloride; and
(iii) 12.5 mg of hydrochlorothiazide
wherein the amlodipine and benazepril are physically separate from each other.

40. A method of treating a hypertensive patient who does not reach a desired target blood pressure following (i) administration of a calcium channel blocker, an ACE inhibitor or diuretic alone, or (ii) administration of a combination of an ACE inhibitor and diuretic or a combination of an ACE inhibitor and calcium channel blocker, comprising administering once a day to said patient a pharmaceutical composition comprising:
(i) 5 mg of amlodipine besylate;
(ii) 40 mg of benazepril hydrochloride; and
(iii) 12.5 mg of hydrochlorothiazide
wherein the amlodipine and benazepril are physically separate from each other.

41. A method of treating a hypertensive patient who does not reach a desired target blood pressure following (i) administration of a calcium channel blocker, an ACE inhibitor or diuretic alone, or (ii) administration of a combination of an ACE inhibitor and diuretic or a combination of an ACE inhibitor and calcium channel blocker, comprising administering once a day to said patient a pharmaceutical composition comprising:
(i) 10 mg of amlodipine besylate;
(ii) 40 mg of benazepril hydrochloride; and
(iii) 12.5 mg of hydrochlorothiazide
wherein the amlodipine and benazepril are physically separate from each other.

42. A method of treating a hypertensive patient who does not reach a desired target blood pressure following (i) administration of a calcium channel blocker, an ACE inhibitor or diuretic alone, or (ii) administration of a combination of an ACE inhibitor and diuretic or a combination of an ACE inhibitor and calcium channel blocker, comprising administering once a day to said patient a pharmaceutical composition comprising:
(iv) 10 mg of amlodipine besylate;
(v) 40 mg of benazepril hydrochloride; and
(vi) 25 mg of hydrochlorothiazide
wherein the amlodipine and benazepril are physically separate from each other.
